(19) United States
(12) Patent Application Publication
(10) Pub. No.: US 2018/0200274 A1
(43) Pub. Date: Jul. 19, 2018

(54) INHIBITORY COMPOSITIONS

(71) Applicant: InQpharm Group Sdn Bhd

(72) Inventors: Pee Win CHONG, Kuala Lumpur (MY); Bee Kwan TAN, Kuala Lumpur (MY)

(21) Appl. No.: 15/529,817

(22) PCT Filed: Nov. 26, 2015
(86) PCT No.: PCT/MY2015/000098

§ 371 Date: May 25, 2017

(30) Foreign Application Priority Data
Nov. 27, 2014 (MY) PI2014703552

(51) Int. Cl. A61K 31/7004; A61K 31/05; A61K 36/87; A61K 45/06; A61P 3/04; A61P 3/10

(52) U.S. Cl. CPC A61K 31/7004; A61K 31/05; A61K 36/87; A61K 2300/00; A61P 3/04; A61P 3/10; A61K 45/06

(57) ABSTRACT

The present invention related to a composition comprising a pentose and one or more polyphenolic compounds, and to the use of said compositions for managing or reducing glucose and/or lipid absorption in a subject, and/or calorie absorption from dietary protein, carbohydrate and/or fat in a subject. The composition may be used to inhibit one or more enzymes selected from α-glucosidase, inhibitory compositions α-amylase, lipase and protease. The composition may be used to treat or prevent obesity and/or metabolic syndrome and/or hyperglycemia and/or hyperlipidemia in a subject.

### TECHNICAL FIELD

The present invention is directed to a composition comprising a pentose and one or more polyphenolic compounds, and to the use of said compositions for managing or reducing blood glucose and/or lipid absorption in a subject, and/or calorie absorption from dietary protein in a subject. The composition may be used to treat or prevent obesity and/or metabolic syndrome in a subject.

### BACKGROUND OF THE INVENTION

Overweight or obesity is a result of chronic imbalance between food consumption and energy expenditure. It can result from a high level of energy intake that exceeds the body's energy expenditure, which may be caused by overeating, inactive or sedentary lifestyle, genetics or family history, medical conditions and/or other factors. This condition is believed to affect over one billion adults worldwide and is rising with excessive intake of refined carbohydrates. Obese or overweight individuals have an increased risk of various types of metabolic syndrome, particularly cardiovascular conditions and type II diabetes. It has been estimated that a 5 kg weight loss over time could account for a 55% reduction in the risk of diabetes (Hamman, R F et al, 2006). Weight reduction has also been associated with a markedly lower risk of cardiovascular diseases (Lavie, Milani & Ventura, 2009). Therefore, body weight management has become a key element of modern healthcare.

In order to combat or prevent obesity or excess body weight, lifestyle changes could be one of the main solutions. However, as physical activity in the developed world continues to decline and lifestyle changes may be difficult to implement, control of calorie intake or absorption has become a more accommodating and effective way to weight management, in order to prevent the further increase of the prevalence of worldwide obesity and its associated medical problems.

Reduction of total caloric intake from diet could be achieved via diet planning, which includes reduction of amount of diet intake, or ingestion of drugs or supplements such as caloric blocker, meal replacement, appetite reducer and others. However, the effectiveness of currently available drugs and supplements for promoting weight control or weight loss is very variable, particularly if they are not used in conjunction with a calorie-restricted diet and exercise regimen.

Therefore, treatments to reduce uptake of dietary fat, carbohydrate and protein are desirable.

### SUMMARY OF THE INVENTION

According to a first aspect, there is provided a composition comprising a pentose and one or more polyphenolic compounds.

According to a second aspect, there is provided a pharmaceutical composition comprising a composition according to the first aspect and a pharmaceutically acceptable carrier and/or excipient and/or diluent. In certain embodiments, the composition is a nutraceutical composition.

According to a third aspect, there is provided a composition according to the first or second aspects for use in inhibiting an enzyme selected from one or more of α-glucosidase, α-amylase, lipase and protease.

According to a fourth aspect, there is provided an in vitro method for inhibiting an enzyme selected from one or more of α-glucosidase, α-amylase, lipase and protease, said method comprising contacting the enzyme with a composition according to the first or second aspect.

According to a fifth aspect, there is provided an in vivo method for inhibiting an enzyme selected from one or more of α-glucosidase, α-amylase, lipase and protease, said method comprising contacting the enzyme with a composition according to the first or second aspect.

According to a sixth aspect, there is provided a method of managing, for example, reducing blood glucose and/or lipid level in a subject, comprising administering to the subject an effective amount of a composition according to the first or second aspect.

According to a seventh aspect, there is provided a method of managing, for example, reducing glucose and/or lipid absorption in a subject, comprising administering to the subject an effective amount of a composition according to the first or second aspect.

According to an eighth aspect, there is provided a method of managing, for example, reducing digestion of dietary protein, carbohydrate and/or fat and absorption of calories from said dietary protein, carbohydrate and/or fat in a subject, comprising administering to the subject an effective amount of a composition according to the first or second aspect, wherein the management or reduction of digestion and absorption of dietary protein is obtained by inhibiting the activity of protease, and/or wherein the management or reduction of digestion and absorption of dietary carbohydrate is obtained by inhibiting the activity of α-glucosidase and α-amylase, and/or wherein the management or reduction of digestion and absorption of dietary fat is obtained by inhibiting the activity of lipase.

According to a ninth aspect, there is provided a composition according to the first or second aspect for use in managing, for example, reducing glucose and/or lipid absorption in a subject.

According to a tenth aspect, there is provided a composition according to the first or second aspect for use in managing, for example, reducing blood glucose and/or lipid level in a subject.

According to an eleventh aspect, there is provided a composition according to the first aspect or second aspect for use in managing, for example, reducing digestion of dietary protein, carbohydrate and/or fat and absorption of calories from said dietary protein, carbohydrate and/or fat in a subject.

According to a twelfth aspect, there is provided a non-therapeutic method of reducing the body weight of a subject, said method comprising administering to the subject an effective amount of a composition according to first or second aspect.

According to a thirteenth aspect, there is provided a composition according to the first or second aspect for use in therapeutically reducing the body weight of a subject.

According to a fourteenth aspect, there is provided a method of reducing the caloric absorption from dietary fats, carbohydrates and/or proteins in a subject, said method comprising administering to the subject an effective amount of a composition according to the first or second aspect.

According to a fifteenth aspect, there is provided a composition according to the first or second aspect for use in reducing the caloric absorption from dietary fats, carbohydrate and/or proteins in a subject.

According to a sixteenth aspect, there is provided a method of treating or preventing obesity and/or metabolic syndrome in a subject, said method comprising administering to the subject an effective amount of a composition according to the first or second aspect.

According to a seventeenth aspect, there is provided a method of treating or preventing hyperglycemia and/or hyperlipidemia in a subject, said method comprising administering to the subject an effective amount of a composition according to the first or second aspect.

According to an eighteenth aspect, there is provided a composition according to the first or second aspect for use in treating or preventing obesity and/or metabolic syndrome in a subject.

According to a nineteenth aspect, there is provided a composition according to the first or second aspect for use in treating or preventing hyperglycemia and/or hyperlipidemia in a subject.

According to a twentieth aspect, there is provided a use of a composition according to the first or second aspect in the manufacture of a medicament for managing, for example, reducing glucose and/or lipid absorption in a subject, or for managing, for example, reducing blood glucose and/or lipid level in a subject, or for treating or preventing obesity and/or metabolic syndrome in a subject, or for treating or preventing hyperglycemia and/or hyperlipidemia in a subject.

According to an twenty first aspect, there is provided a use of a composition according to the first or second aspect in the manufacture of a medicament for managing, for example, reducing digestion of dietary protein, carbohydrate and/or fat and absorption of calories from said dietary protein, carbohydrate or fat in a subject.

### DETAILED DESCRIPTION OF THE INVENTION

The present invention is based on, at least in part, the surprising finding that a combination of a pentose (for example, L-arabinose) and one or more polyphenolic compounds (e.g., plant-derived tannins) can be used to manage, e.g., reduce glucose (sugar) and/or lipid absorption in a subject, and/or digestion of dietary protein, carbohydrate and/or fat and absorption of calories from said dietary protein, carbohydrate and/or fat in a subject. Without wishing to be bound by theory, it is believed that the composition inhibits the activity of enzymes such as α-glucosidase, α-amylase, lipase and protease (e.g., trypsin) in the gastrointestinal tract.

The composition comprises a pentose (a monosaccharide with five carbon atoms), for example, aldopentose (having an aldehyde functional group at position 1 of the carbon chain) and/or ketopentose (having a ketone functional group at position 2 or 3 of the carbon chain. In certain embodiments, the pentose is a L-pentose, for example, L-aldopentose or L-ketopentose. In certain embodiments, the pentose is a D-pentose, for example, D-aldopentose or D-ketopentose. In certain embodiments, the pentose is an aldopentose selected from L-arabinose, D-lyxose, L-lyxose, D-ribose, L-ribose, D-xylose, L-xylose, and mixtures thereof. In certain embodiments, the pentose is selected from L-arabinose, L-lyxose, L-ribose, L-xylose, and mixtures thereof. In certain embodiments, the pentose is L-arabinose. Thus, in certain embodiments, the composition comprises L-arabinose. In certain embodiments, the L-arabinose is plant-derived. In certain embodiments, the L-arabinose is derived from the plant Zea mays, for example, corn or maize, or is derived from gum Arabic. Other sources of L-arabinose include sugarcane bagasse, wood base (cellulose) substances such as wheat, rye, rice and pectic substances from beet and apple pulps, as well as some plant gums.

The composition of the present invention further comprises one or more polyphenolic compounds. In certain embodiments, the one or more polyphenolic compounds are plant-derived. In certain embodiments, the one or more plant-derived polyphenolic compounds are plant-derived tannins. In certain embodiments, the plant-derived tannins are condensed tannins and/or hydrolysable tannins. Condensed tannins are also known as proanthocyanidins, which yield anthocyanidins. Hydrolysable tannins include gallotannins and ellagitannins.

In certain embodiments, plant-derived tannins are derived from the plant Vitis spp., for example, Vitis vinifera an/or Vitis rotundifolia. In certain embodiments, the plant-derived tannins are derived from Vitis vinifera. In certain embodiments, the plant-derived tannins are comprised within (i.e., are components of) grapes, including grape step, skin, pulps and seed. In certain embodiments, the plant-derived tannins are comprised within grape marc extract. Grape marc extract comprises the solid remains of grapes after pressing, e.g., for juice. It typically contains skins, pulp, seeds and stems of the fruit. Grape marc extract is a brown and astringent powder produced from the aqueous extraction of grape marc. In certain embodiments, the grape marc extract is derived from Chardonnay, Grenache, Syrah, Carignan, Mourvédre, Counoise and/or Alicante.

The grape marc extract may comprise both tannins and other types of polyphenolic compounds. In certain embodiments, the grape marc extract (and, thus, the composition) contains both condensed tannins and hydrolysable tannins. In certain embodiments, condensed tannins possess higher activity in the digestive enzymes-binding effects relative to hydrolysable tannins.

In certain embodiments, the grape marc extract comprises at least about 1% w/w tannins, i.e., based on the total weight of the grape marc extract, for example, at least about 2% w/w tannins, or at least about 3% w/w tannins, or at least about 5% w/w tannins, or at least about 10% w/w tannins, or at least about 15% w/w tannins, or at least about 20% w/w tannins, or at least about 25% w/w, or at least about 30% w/w tannins. In certain embodiments, the grape marc extract comprises from about 20% w/w to about 40% w/w tannins, for example, from about 25% w/w to about 40% w/w tannins, or from about 30% w/w to about 40% w/w tannins, or from about 32% w/w to about 38% w/w tannins, or from about 33% w/w to about 35% w/w tannins. In certain embodiments, the grape marc extract comprises no more than about 50% w/w tannins, for example, no more than about 45% w/w tannins, or no more than about 40 w/w tannins, or no more than about 35% w/w tannins.

Hereafter, the invention may tend to be discussed in terms of L-arabinose, and in relation to compositions comprising L-arabinose and the preparation thereof. The invention should not be construed as being limited to such embodiments, and extends to other types of pentose as described above.

In certain embodiments, the composition comprises L-arabinose and grape marc extract. In certain embodiments, the weight ratio of a L-arabinose to grape marc extract is from about 95:5 to about 40:60, for example, from about 90:10 to about 40:60, or from about 85:15 to about 40:60, or from about 80:20 to about 40:60, or from about 75:25 to about 40:60, or from about 70:30 to about 40:60, or from about 65:45 to about 40:60, or from about 60:40 to about 40:60, or from about 55:45 to about 40:60, or from about 50:50 to about 40:60, or from about 50:50 to about 40:60, or from about 48:52 to about 40:60, or from about 48:52 to about 42:58 or from about 47:53 to about 43:57, or from about 47:53 to about 44:56. In certain embodiments, the weight ratio of L-arabinose and grape marc extract is approximately 46:54. In such embodiments, the grape marc extract may comprise from about 25% w/w to about 40% w/w tannins, for example, from about 30% w/w to about 40% w/w tannins, or from about 32% w/w to about 38% w/w tannins, or from about 33% w/w to about 35% w/w tannins.

In certain embodiments, the composition comprises at least about 20% w/w L-arabinose and/or at least about 2% w/w grape marc extract, based on the total weight of the composition. In certain embodiments, the composition comprises at least about 30% w/w L-arabinose and/or at least about 3% w/w grape marc extract, or at least about 40% w/w L-arabinose and/or at least about 4% w/w grape marc extract, or at least about 30% w/w L-arabinose and/or at least about 10% w/w grape marc extract, or at least about 30% w/w L-arabinose and/or at least about 15% w/w grape marc extract, and/or at least about 30% w/w L-arabinose and/or at least about 20% w/w grape mar extract, and/or at least about 30% w/w L-arabinose and/or at least about 25% w/w grape marc extract, or at least about 30% w/w L-arabinose and at least about 30% w/w grape marc extract. In such embodiments, the grape marc extract may comprise from about 25% w/w to about 40% w/w tannins, for example, from about 30 w/w to about 40% w/w tannins, or from about 32% w/w to about 38% w/w tannins, or from about 33% w/w to about 35% w/w tannins.

In certain embodiments, the total amount of L-arabinose and grape marc extract constitutes no more than about 95% w/w of the composition, for example, no more than 90% w/w of the composition, no more than about 80% w/w of the composition, or no more than about 70% w/w of the composition, or no more than about 60% w/w of the composition, or no more than about 50% w/w of the composition. In such embodiments, the weight ratio of L-arabinose to grape marc extract may be from about 95:5 to about 40:60, for example, from about 90:10 to about 40:60, or from about 85:15 to about 40:60, or from about 80:20 to about 40:60, or from about 75:25 to about 40:60, or from about 70:30 to about 40:60, or from about 65:45 to about 40:60, or from about 60:40 to about 40:60, or from about 55:45 to about 40:60, or from about 50:50 to about 40:60, or from about 50:50 to about 40:60, or from about 48:52 to about 40:60, or from about 48:52 to about 42:58 or from about 47:53 to about 43:57, or from about 47:53 to about 44:56. In such embodiments, the grape marc extract may comprise from about 25% w/w to about 40% w/w tannins, for example, from about 30% w/w to about 40% w/w tannins, or from about 32% w/w to about 38% w/w tannins, or from about 33% w/w to about 35% w/w tannins.

In certain embodiments, the composition further comprises dietary fibre of plant and/or non-plant origin. The term “dietary fibre” used herein has its normal meaning for this term. It is generally regarded as the indigestible portion of food derived from plants. Typically, there are two main components of dietary fibre: soluble fibre, which dissolves in water, and insoluble fibre, which does not dissolve in water. Soluble fibres include chitosan, gum acacia, guar gum, low-methoxy and high-methoxy pectin, oat and/or barley beta glucans, carrageenan, psyllium, cyclodextrin, and derivatives thereof. Insoluble fibres includeoat hull fibre, pea hull fibre, soy hull fibre, soy cotyledon fibre, sugar beet fibre, cellulose, corn bran and derivatives thereof. In certain embodiments, the dietary fibre is derived from Abelmoschus app. In certain embodiments, the dietary fibre is okra powder or fibre. In certain embodiments, the composition comprises from about 0.1% to about 90% by weight of dietary fibre, for example, from about 1% to about 80% by weight, or from about 1% to about 70% by weight, or from about 1% to about 60% by weight, or from about 1% to about 50% by weight, or from about 5% to about 50% by weight, or from about 10% to about 50% by weight, or from about 20% to about 50% by weight by weight of dietary fibre, based on the total weight of the composition.

In certain embodiments, the composition further comprises other biologically active agents, for example, biologically active agents suitable for treating obesity and/or metabolic diseases such as metabolic syndrome. In certain embodiments, the biologically active agent is selected from the group consisting of absorption-altering agents, including lipase inhibitors, e.g., orlistat and cetilistat, fat binders, e.g., dehydrated Opuntia ficus indica cladode powder, alpha amylase inhibitors, e.g., white kidney bean extract, and alpha glucosidase inhibitors, e.g., acarbose and tannins; appetite-altering agents, including pharmaceutical agents, e.g., sibutramine, phentermine, diethylpropion, rimonabant, benzphetamine and nutraceutical agent, e.g., potato extract and protein; metabolism-altering agents such as monoxidine, green tea extract, Garcinia cambogia extract, Citrus aurantum extract; cholesterol-lowering agents including statins, e.g., simvastatin, atorvastatin, lovastatin, pravastatin and rosuvastatin etc., fibrates (gemfibrosil, bezafibrate, fenofibrate or ciprofibrate), bile acid sequestrants eg colestipol, cholestyramine and nutraceuticals, e.g., plant sterol or any combination thereof. In certain embodiments, the biologically active agent or agents are present in the composition in an amount ranging from about 0.001 wt. % to about 50 wt. %, based on the total weight of the composition, for example, about 0.1 wt. % to about 15 wt. %, or from about 0.5 wt. % to about 10 wt. %, or from about 0.5 wt. % to about 5 wt. %, or from about 0.1 wt. % to about 3 wt. %, or from about 0.1 wt. % to about 2 wt. %, or from about 0.1 wt. % to about 1 wt. %, or from about 0.001 wt. % to about 5 wt. %, or from about 0.001 wt. % to about 2 wt. %, or from about 0.001 wt. % to about 1 wt. %, or from about 0.001 wt. % to 20 about 0.5 wt. %, or from about 0.001 wt. % to about 0.1 wt. %, or from about 0.001 wt. % to about 0.01 wt. %.

In certain embodiments, the composition further comprises a nutrient ingredient selected from the group consisting of vitamins and minerals, and combinations thereof. The vitamin may be any one or more of vitamin A, vitamin D, vitamin E, vitamin K, thiamine, riboflavin, pyridoxine, vitamin B12, caratenoids (including beta-carotene, zeaxanthin, lutein and lycopene), niacin, folic acid, pantothenic acid, biotin, vitamin C, choline, inositol, and salts and derivatives thereof. The mineral may be any one or more of calcium, phosphorous, magnesium, iron, zinc, manganese, copper, cobalt, boron, iodine, sodium, potassium, molybdenum, selenium, chromium, fluorine and chloride. If present, in certain embodiments, the composition comprises from about 0.0001% to about 50% by weight of vitamin(s) and/or mineral(s), based on the total weight of the composition, for example, from about 0.01% to about 45% by weight, from about 0.1% to about 40% by weight, or from about 0.5% to about 30% by weight, or from about 0.5% to about 20% by weight, or from about 0.5% to about 10% by weight, or from about 0.5% to about 5%, or 20 from about 0.5% to about 3%, or from about 0.1% to about 2%, or from about 0.1 to about 1% of vitamin(s) and/or mineral(s), based on the total weight of the composition. In certain embodiments, the composition comprises from about 0.0001% to about 5 wt. %, for example, from about 0.0001% to about 2 wt. %, or from about 0.0001% to about 1 wt. %, or from about 0.0001% to about 0.5 wt. %, or from about 0.0001% to about 0.1 wt. %, or from about 0.0001% to about 0.01 wt. % by weight of vitamin(s) and/or mineral(s), based on the total weight of the composition.

The composition of the present invention may be administered in the form of a composition comprising any suitable additional component. The composition may, for example, be a pharmaceutical composition (medicament), suitably for oral administration (e.g. tablet, capsule, powder, liquid, and the like). The composition may alternatively be a nutraceutical composition, for example, a foodstuff, food supplement, dietary supplement, health supplement, meal replacement product, beverage, beverage supplement, food additive, animal feed or feed additive.

The term “pharmaceutical composition” or “medicament” in the context of this invention means a composition comprising (a pharmaceutically effective amount of) the L-arabinose and one or more polyphenolic compounds and additionally one or more pharmaceutically acceptable carriers and/or excipients. The composition may further contain ingredients selected from, for example, diluents, adjuvants, excipients, vehicles, preserving agents, fillers, binders, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of solid preparations including tablets, capsules, drageés, lozenges, granules, powders, pellets and cachets; and liquid preparations including elixirs, syrups, suspensions, sprays, emulsions and solutions. Techniques and formulations generally may be found in Remington, The Science and Practice of Pharmacy, Mack Publishing Co., Easton, Pa., latest edition.

In solid dosage forms of the invention for oral administration, the active ingredient(s) may be mixed with one or more pharmaceutically acceptable carriers, such as dicalcium phosphate, and/or any of the following: diluents, fillers or extenders, such as starches, lactose, sucrose, glucose, mannitol, microcrystalline cellulose and/or silicic acid; binders, such as, for example, hydroxypropylcellulose, hypromellose, hydroxypropyl methylcellulose, carboxymethylcellulose, gelatine, polyvinyl pyrrolidones, polyvinyl acetate, sucrose and/or acacia; disintegrating agents, such as starch, for example, potato or tapioca starch, starch derivatives such as sodium starch glycolate, crospolyvinylpyrollidone, calcium carbonate, croscarmellose sodium, alginic acid, and certain silicates; lubricants, such as talc, calcium stearate, magnesium stearate, stearic acid, sodium sulfate stearyl fumarate, solid polyethylene glycols, solubiliser such as sodium lauryl sulfate, flavouring and colouring agents and mixtures thereof.

Tablets, and other solid dosage forms of the pharmaceutical compositions of the invention, may optionally be prepared with coatings and shells, such as enteric coatings and other coatings well known in the pharmaceutical-formulation art. They may also be formulated so as to provide slow or controlled release of the active ingredient(s) therein using, for example, natural and synthetic polymers such as hydroxypropylmethyl cellulose methacrylates respectively, in varying proportions to provide the desired release profile, other polymer matrices, liposomes and/or microspheres may also be used. These compositions may also optionally contain colourants and/or opacifying agents and may be of a composition such that they release the active ingredient(s) only, or preferentially, in a certain portion of the gastrointestinal tract, optionally, in a delayed manner.

In certain embodiments, the pharmaceutical composition comprises pharmaceutically acceptable carriers and/or excipients selected from one or more of binder, diluent, lubricant and coating agent.

In certain embodiments, pharmaceutical composition comprises no more than about 50% w/w of pharmaceutically acceptable carrier and/or excipient, for example, no more than about 45% w/w of pharmaceutically acceptable carrier and/or excipients, or no more than about 40% of w/w pharmaceutically acceptable carrier and/or excipients, or no more than about 35% w/w of pharmaceutically acceptable carrier and/or excipients. In certain embodiments, the pharmaceutical composition comprises at least about 1% w/w, or at least about 10% w/w., or at least about 15% w/w, or at least about 20% w/w, or at least about 25% w/w, or at least about 30% w/w of pharmaceutically acceptable carrier and/or excipients.

Liquid form preparations include solutions, suspensions, and emulsions, for example, water or water-propylene glycol solutions for oral administration. Liquid preparations can also be formulated in solution in aqueous polyethylene glycol solution. In certain embodiments, the active ingredient(s), i.e., L-arabinose and grape marc extract, may be mixed with one or more pharmaceutically acceptable carriers, such as water and/or any of the following: solvent such as propylene glycol, alcohol; humectant such as glycerol; sweeteners such as liquid glucose, corn syrup and sucrose; artificial sweeteners such as aspartame, stevia and sucralose; preservatives such as benzoates and parabens; viscosity modifiers/thickeners such as gums and alginates; buffering agents; flavouring agents and colouring agents.

Also included are solid form preparations, for example, tablets, granules and powder, which are intended to be converted, shortly before use, to liquid form preparations for oral administration. Such liquid forms include solutions, suspensions, and emulsions. These particular solid form preparations are most conveniently provided in unit dose form and as such are used to provide a single liquid dosage unit. Alternately, sufficient solid may be provided so that multiple individual liquid doses may be reconstituted when required, by measuring predetermined volumes of the solid form preparation as with a spoon, or other measuring device. The solid form preparations intended to be converted to liquid form may contain, in addition to the active material, flavourings, colourants, stabilizers, buffers, artificial and natural sweeteners, dispersants, thickeners, solubilising agents, and the like. The liquid utilized for preparing the liquid form preparation may be water, isotonic water, juices, milk, ethanol, and the like as well as mixtures thereof.

The terms “food”, “foodstuff”, “food supplement”, “dietary supplement”, “health supplement”, “meal replacement product”, “beverage” and “beverage supplement” used herein have the normal meanings for those terms, and are not restricted to pharmaceutical preparations. Other composition forms are also included within the present invention. These may, for example, include, a foodstuff precursor such as a rehydratable powder or a beverage precursor such as a powder dispersible in water, milk or other liquid.

Also included are solid form preparations which are intended to be combined with a food or foodstuff before oral consumption. The solid form preparations may be mixed into the food or foodstuff or applied to the food or foodstuff, e.g., by sprinkling onto the food or foodstuff. Such solid forms include powders, granules, pellets and the like.

Such food of foodstuffs include, without limitation, prepared meals (cooked or fresh), soup, dairy based products (e.g., yoghurt, cream, crème-fraîche), flour based products such as bread and pasta, snack or convenience items such as snack bars (e.g., chocolate bars), confectionary products, and the like.

In certain embodiments, the food or foodstuff, and the like, comprises from about 0.1 wt. % to about 50 wt. % of the composition of the invention described herein, based on the total weight of the food or foodstuff, for example, from about 0.1 wt. % to about 40 wt. %, or from about 0.1 wt. % to about 30 wt. %, or from about 0.1 wt. % to about 20 wt. %, or from about 0.1 wt. % to about 15 wt. %, or from about 0.1 wt. % to about 10 wt. %, or from about 0.1 wt. % to about 8 wt. %, or from about 0.1 wt. % to about 6 wt. %, or from about 0.1 wt. % to about 4 wt. %, or from about 0.1 wt. % to about 2 wt. % of the composition of the invention described herein. In certain embodiments, the food or foodstuff, and the like, comprise at least about 0.2 wt. % of the compositions of the invention described herein, based on the total weight of the food or foodstuff, for example, at least about 0.5 wt. %, or at least about 1 wt. %, or at least about 5 wt. % of the composition of the invention described herein.

In certain embodiments, the composition is orally administered daily to the subject. Without wishing to be bound by theory, it is believed that the composition inhibits the activity of enzymes such as α-glucosidase, α-amylase, lipase and trypsin in the gastrointestinal tract, and thus achieves a reduction in calorie absorption from dietary fats, carbohydrates and/or protein. The composition is desirably administered prior to, with, or after a meal, depending on the nature of the oral dosage form; for example, a capsule or powder may be administered approximately 15 minutes to 60 minutes before or after a meal, for example, 15 minutes to 30 minutes before or 30 minutes to 45 minutes after a meal.

The amount of composition administered may be varied depending upon the requirements of the subject and the amount of macronutrients, i.e., fats, carbohydrates and protein in the food or diet being consumed. For therapeutic applications, the amount of composition administered may be varied depending upon the requirements of the subject, the severity of the condition being treated, and the amount of calories in the food or diet being consumed. Determination of the proper amount/dosage for a particular situation is within the skill of the art. For example, for therapeutic applications a physician or veterinarian having ordinary skill in the art can readily determine and prescribe the effective amount of the pharmaceutical composition required. The total daily amount/dosage may be divided and administered with meals in portions during the day if desired.

In general, a suitable daily dose of a composition according to the invention will be that amount of the composition which is the lowest dose effective to produce the desired effect, for example, a therapeutic effect, and/or to reduce blood sugar and/or lipid absorption. It is contemplated that a wide range of doses may be used, due to the non-toxic nature of the composition. For example, the dose of the composition may be up to 15 g per day, with the weight ratio of L-arabinose and grape marc extract from about 95:5 to about 40:60, for example, up to about 10 g per day, or up to about 5 g per day. In certain embodiments, the doses of the composition is in the range of 100 mg to about 3 g per day, with the weight ratio of L-arabinose and grape marc extract from about 95:5 to about 40:60, which may be administered as two or three or more sub-doses administered separately at appropriate intervals (e.g., after each meal) throughout the day, optionally in unit dosage forms. In certain embodiments, the dose of the composition may be from about 200 mg to about 3 g of each component per day, for example, from about 500 mg to about 3 g of each component per day, or from about 750 mg to about 2.5 g of each component per day, or from about 1000 mg to about 2000 mg of each component per day. In certain embodiments, the composition may be administered two or three times a day, optionally before, with, or after a meal. In certain embodiments, the dose per meal is no more than about 5 g of the composition, for example, no more than about 3 g of the composition, for example, no more than about 2.5 g of the composition.

As set forth in the preceding description, the weight ratio of L-arabinose to grape marc extract may be from about 95:5 to about 40:60. Therefore, the daily dose of L-arabinose can be in the range of 250 mg to 10 g per day; whereas the daily dose of grape marc extract can be in the range of 50 mg to 2 g per day. For example, the recommended daily dosage of the composition can be 2.6 g, i.e., the composition can contain 2400 mg of L-arabinose and 180 mg of grape marc extract. In another example, the composition can contain 920 mg of L-arabinose and 1080 mg of grape marc extract, for a total dosage of 2 g per day.

The compositions described herein may be used in various therapeutic applications.

The expression “treating or preventing” and analogous terms used herein refers to all forms of healthcare intended to remove or avoid the disorder or to relieve its symptoms, including preventive and curative care, as judged according to any of the tests available according to the prevailing medical practice. An intervention that aims with reasonable expectation to achieve a particular result but does not always do so is included within the expression “treating or preventing”. An intervention that succeeds in slowing or halting progression of a disorder is included within the expression “treating or preventing”.

The expression “susceptible to” and analogous terms used herein refers particularly to individuals at a higher than normal risk of developing a disease, for example, obesity and/or metabolic syndrome, as assessed using the known risk factors for the individual or disease, e.g., obesity/metabolic syndrome. Such individuals may, for example, be categorised as having a substantial risk of developing the disease, e.g., obesity and/or metabolic syndrome, to the extent that medication would be prescribed and/or special dietary, lifestyle or similar recommendations would be made to that individual.

In certain embodiments, the subject is a human. In other embodiments, the subject is a mammal other than a human, as described above.

In certain embodiments, the composition according to first and second aspects of the present invention may be used to inhibit enzymes, for example, one or more digestive enzymes.

Mammalian α-glucosidase is a glucosidase that acts upon 1,4 α-bonds. It as an enzyme which breaks down carbohydrates (such as starch) and disaccharides (such as table sugar) to glucose. Carbohydrates and disaccharides are normally converted into simple sugars, which can be absorbed through the intestine. Thus, reducing (i.e., inhibiting) the activity of α-glucosidase would be expected to reduce sugar absorption and thus would be expected to reduce blood sugar levels.

Mammalian α-amylase is an enzyme which hydrolyses α-bonds of large α-linked polysaccharides, such as starch or other complex carbohydrates, producing glucose and maltose, which can be absorbed through the intestine. Thus, reducing (i.e., inhibiting) the activity of α-amylase would be expected to reduce sugar absorption, and thus would be expected to reduce blood sugar levels.

Mammalian lipase is an enzyme which catalyzes the hydrolysis of lipids and, thus aids in the digestion of dietary fats. Thus, reducing (i.e., inhibiting) the activity of lipase would be expected to reduce absorption of dietary fats and, thus, reduce lipid level.

Mammalian protease (for example, trypsin, pepsin and/or chymotrypsin) is a family of enzymes which hydrolyse proteins, breaking down the proteins into smaller peptides, which may be further hydrolysed into amino acids, rendering them available for absorption into the blood stream. Thus, reducing (i.e., inhibiting) the activity of a protease (for example, trypsin, pepsin and/or chymotrypsin) would be expected to reduce absorption of calories from dietary protein, which may be desirable. In certain embodiments, the protease is a member of the serine protease family. In certain embodiments, the protease is a member of the aspartate protease family.

Without wishing to be bound by theory, it is believed that the combination of L-arabinose and/or the one or more polyphenolic compounds (e.g., tannins derived from grape marc extract) binds with α-amylase and α-glucosidase, inhibiting the activity of the enzymes. Unexpectedly, in certain embodiments it has been found that the combination of L-arabinose and one or more polyphenolic compounds (e.g., tannins derived from grape marc extract) provides an increase in the inhibition of α-amylase and/or α-glucosidase. The combination of L-arabinose and/or the one or more polyphenolic compounds (e.g., tannins derived from grape marc extract) may also advantageously inhibit the activity of lipase and protease (e.g., trypsin). Thus, in certain embodiments, the compositions according to the first and second aspects of the present invention may be used to simultaneously inhibit the activity of α-amylase, α-glucosidase, lipase and protease (e.g., trypsin). Thus, in certain embodiments, the composition of the first aspect of the present invention is referred to as an enzyme-inhibitor composition, i.e., has the function of inhibiting enzymatic activity. The combination of L-arabinose and one or more polyphenolic compounds (e.g., tannins from grape marc extract) may be present in suitable, relative amounts, as described herein such that the combination functions to inhibit the activity of one or more of -amylase, α-glucosidase, lipase and protease (e.g., trypsin, pepsin or chymotrypsin).

The inhibitory properties of the compositions according to the first and second aspects of the present invention may be determined in vivo or in vitro. In vitro methods for determining the inhibitory of the compositions of the present invention are described in the Examples section below. For example, an inhibitory property of the composition may be expressed in terms of the amount of the composition required to inhibit the activity of a certain amount enzyme of by 50% (i.e., an IC50 expressed in μg or mg of the composition per ml of enzyme).

In certain embodiments, the composition has an IC50 for α-amylase of less than about 250 μg/ml, for example, less than about 200 μg/ml, or less than about 150 μg/ml, or less than about 100 μg/ml, or less than about 50 μg/ml, or less than about 25 μg/ml, or less than about 15 μg/ml.

In certain embodiments, the composition has an IC50 for α-glucosidase of less than about 800 μg/ml, for example, less than about 750 μg/ml, or less than about 650 μg/ml, or less than about 600 μg/ml, or less than about 500 μg/ml.

In certain embodiments, the composition has an IC50 for trypsin of less than about 250 μg/ml, for example, less than about 200 μg/ml, or less than about 100 μg/ml, or less than about 50 μg/ml.

In certain embodiments, the composition has an IC50 for lipase of less than about 100 μg/ml, for example, less than about 75 μg/ml, or less than about 50 μg/ml, or less than about 25 μg/ml, or less than about 15 μg/ml, or less than about 10 μg/ml.

In certain embodiments, the composition has:
an IC50 for α-amylase of less than about 15 μg/ml, and
an IC50 for α-glucosidase of less than about 600 μg/ml, and
an IC50 for trypsin of less than about 50 μg/ml, and
an IC50 for lipase of less than about 10 μg/ml.

According to certain embodiments, there is provided an in vivo method for inhibiting an enzyme selected from one or more of α-glucosidase, α-amylase, lipase and trypsin, said method comprising contacting the enzyme with a composition according to the first and second aspects of the present invention, i.e., a composition comprising a combination of L-arabinose and one or more polyphenolic compounds (e.g., tannins derived from grape marc extract).

According to certain embodiments, there is provided an in vitro method for inhibiting an enzyme selected from one or more of α-glucosidase, α-amylase, lipase and trypsin, said method comprising contacting the enzyme with a composition according to first and second aspects of the present invention, i.e., a composition comprising a combination of L-arabinose and one or more polyphenolic compounds (e.g., tannins derived from grape marc extract).

Further, because the composition comprising the combination of L-arabinose and one or more polyphenolic compounds (e.g., tannins derived from grape marc extract) has been found to inhibit the activity of digestive enzymes, the compositions according to certain embodiments is used in a method of managing, for example, reducing, blood glucose levels in a subject. The method comprises administering to the subject an effective amount of a composition according to certain embodiments described herein. In certain embodiments, the method is a method of reducing blood sugar levels in a subject. In certain embodiments, the management or reduction of blood glucose levels is obtained by inhibiting the activity of α-glucosidase and α-amylase, and reducing glucose absorption.

Further, the compositions according to certain embodiments may be used in a method of managing, for example, reducing, blood lipid levels in a subject. The blood lipid includes total cholesterol, LDL cholesterol, HDL cholesterol and trigylcerides. The method comprises administering to the subject an effective amount of a composition according to certain embodiments described herein. In certain embodiments, the method is a method of reducing blood lipid levels in a subject. In certain embodiments, the management or reduction of lipid levels are obtained by inhibiting the activity of lipase, and reducing lipid absorption. In certain embodiments, the method includes binding dietary fat and, thus, reducing the fat absorption. In such embodiments, the composition may additionally comprise a dietary fibre such as okra powder or fibre having dietary fat-binding properties.

Further, the compositions according to certain embodiments may be used in a method of managing, for example, reducing, amino acid absorption from protein consumed in the diet of a subject. The method comprises administering to the subject an effective amount of a composition according to certain embodiments described herein. In certain embodiments, the method is a method of reducing the absorption of calories from protein consumed in the diet of a subject. In certain embodiments, the management or reduction of absorption of calories from dietary protein is obtained by inhibiting the activity of a protease, such as trypsin, pepsin or chymotrypsin.

The compositions according to certain embodiments may be used in a method of managing, for example, reducing, the absorption of calories from dietary carbohydrate and/or fat consumed in the diet of a subject. In certain embodiments, the management or reduction of absorption of calories from said dietary carbohydrate and/or fat is obtained by inhibiting the activity of α-glucosidase and α-amylase, and lipase, respectively.

In accordance with the therapeutic methods and applications of the present invention described herein, the composition described herein (i.e., L-arabinose and a/the source of one or more polyphenolic compounds, such as grape marc extract) is administered in an effective amount such that glucose and/or lipid absorption, and/or the absorption of calories from dietary protein is reduced, and thus blood sugar, lipid levels and calorie intake are reduced. An effective amount will be understood to be an amount which is effective to reduce sugar, lipid and/or calories absorption, i.e., and which may produce a therapeutic effect. An effective amount includes any of the doses, dosages or dosage regimens described above, each of which pertains to the amount of L-arabinose and the source of the one or more polyphenolic compounds.

In certain embodiments, in each of the therapeutic applications described above, the total amount of L-arabinose and grape marc extract (i.e., the source of the one or more plant-derived tannins) constitutes no more than 90% w/w of the composition, for example, no more than about 80% w/w of the composition, or no more than about 70% w/w of the composition, or no more than about 60% w/w of the composition, or no more than about 50% w/w of the composition. In such embodiments, the weight ratio of L-arabinose to grape marc extract may be from about 95:5 to about 40:60, for example, from about 90:10 to about 40:60, or from about 85:15 to about 40:60, or from about 80:20 to about 40:60, or from about 75:25 to about 40:60, or from about 70:30 to about 40:60, or from about 65:45 to about 40:60, or from about 60:40 to about 40:60, or from about 55:45 to about 40:60, or from about 50:50 to about 40:60, or from about 50:50 to about 40:60, or from about 48:52 to about 40:60, or from about 48:52 to about 42:58 or from about 47:53 to about 43:57, or from about 47:53 to about 44:56. In such embodiments, the grape marc extract may comprise from about 25% w/w to about 40% w/w tannins, for example, from about 30% w/w to about 40% w/w tannins, or from about 32% w/w to about 38% w/w tannins, or from about 33% w/w to about 35% w/w tannins.

Obesity is a medical condition in which excess body fat has accumulated to the extent that it may have an adverse effect on health, leading to reduced life expectancy and/or increased health problems. Subjects are considered obese when their body mass index (BMI), a measurement obtained by dividing a person's weight in kilograms by the square of the person's height in metres, exceeds 30 kg/m² or greater.

Obesity increases the likelihood of various diseases, particularly heart disease, type 2 diabetes, obstructive sleep apnea, certain types of cancer, and osteoarthritis.

BMI is calculated by dividing the subject's mass by the square of his or her height, typically expressed in metric units:

BMI=weight in kilograms/(height in metres)²

The most commonly used definitions, established by the World Health Organization (WHO) in 1997 and published in 2000, provide the values listed in the table below.

| BMI | Classification |
|---|---|
| <18.5 | underweight |
| 18.5-24.9 | normal weight |
| 25.0-29.9 | overweight |
| 30.0-34.9 | class I obesity |
| 35.0-39.9 | class II obesity |
| ≥40.0 | class III obesity |

Metabolic syndrome is a combination of medical disorders that, when occurring together, increase the risk of a subject developing diseases such as cardiovascular disease and diabetes. Metabolic syndrome is also known as metabolic syndrome X, cardiometabolic syndrome, syndrome X, insulin resistance syndrome, Reaven's syndrome (named for Gerald Reaven), and CHAOS (in Australia).

There are a number of different definitions for metabolic syndrome, as follows:

The International Diabetes Federation consensus worldwide definition of metabolic syndrome (2006) is: central obesity (defined as waist circumference with ethnicity-specific values) and any two of the following:
raised triglycerides: >150 mg/dL (1.7 mmol/L), or specific treatment for this lipid abnormality
reduced HDL cholesterol: <40 mg/dL (1.03 mmol/L) in males, <50 mg/dL (1.29 mmol/L) in females, or specific treatment for this lipid abnormality
raised blood pressure (BP): systolic BP>130 or diastolic BP>85 mm Hg, or treatment of previously diagnosed hypertension
raised fasting plasma glucose (FPG): >100 mg/dL (5.6 mmol/L), or previously diagnosed type 2 diabetes

If a subject's BMI is greater than 30 kg/m², central obesity can be assumed and waist circumference does not need to be measured.

The World Health Organization criteria (1999) require the presence of any one of diabetes mellitus, impaired glucose tolerance, impaired fasting glucose or insulin resistance, and two of the following:
blood pressure: ≥140/90 mm Hg
dyslipidemia: triglycerides (TG): ≥1.695 mmol/L and high-density lipoprotein cholesterol (HDL-C)≤0.9 mmol/L (male), ≤1.0 mmol/L (female)
central obesity: waist:hip ratio >0.90 (male); >0.85 (female), or body mass index >30 kg/m²
microalbuminuria: urinary albumin excretion ratio ≥20 μg/min or albumin:creatinine ratio ≥30 mg/g

The European Group for the Study of Insulin Resistance (1999) requires insulin resistance defined as the top 25% of the fasting insulin values among non-diabetic individuals and any two or more of the following:
central obesity: waist circumference ≥0.94 cm (male), ≥80 cm (female)
dyslipidemia: TG≥2.0 mmol/L and/or HDL-C<1.0 mmol/L or treated for dyslipidemia
hypertension: blood pressure ≥140/90 mmHg or antihypertensive medication
fasting plasma glucose ≥6.1 mmol/L

The US National Cholesterol Education Program Adult Treatment Panel III (2001) requires at least three of the following:
central obesity: waist circumference ≥102 cm or 40 inches (male), ≥88 cm or 36 inches (female)
dyslipidemia: TG≥1.7 mmol/L (150 mg/dl)
dyslipidemia: HDL-C<40 mg/dL (male), <50 mg/dL (female)
blood pressure ≥130/85 mm Hg, or treated for hypertension
fasting plasma glucose ≥0.1 mmol/L (110 mg/dl)

In certain embodiments, metabolic syndrome is as defined according to the International Diabetes Federation consensus worldwide definition of metabolic syndrome (2006).

In certain embodiments, metabolic syndrome is as defined according to The World Health Organization criteria (1999).

In certain embodiments, metabolic syndrome is as defined according to The European Group for the Study of Insulin Resistance (1999).

In certain embodiments, metabolic syndrome is as defined according to The US National Cholesterol Education Program Adult Treatment Panel III (2001).

In accordance with the therapeutic methods and applications of the present invention described herein, the composition described herein (i.e., L-arabinose and a/the source of one or more polyphenolic compounds, such as grape marc extract) is administered in an effective amount such that obesity and/or a metabolic disease (e.g., metabolic syndrome) is treated or prevented. An effective amount will be understood to be an amount which is effective to treat or prevent obesity and/or a metabolic disease (e.g., metabolic syndrome), i.e., to produce a therapeutic effect. An effective amount includes any of the doses, dosages or dosage regimens described above, each of which pertains to the amount of L-arabinose and the source of the one or more polyphenolic compounds.

Hyperglycemia, or high blood sugar refers to a condition in which the glucose circulates in the blood plasma is of an excessive amount. This generally happens when a glucose level is higher than 11.1 mmol/l (200 mg/dl), but symptoms may not start to become noticeable until even higher values such as 15-20 mmol/l (˜250-300 mg/dl). According to the American Diabetes Association guidelines, a subject with a consistent range between ˜5.6 and ˜7 mmol/l (100-126 mg/dl) of blood glucose is considered hyperglycemic, while above 7 mmol/l (126 mg/dl) is generally held to have diabetes. Chronic levels exceeding 7 mmol/l (125 mg/dl) can produce organ damage.

Hyperlipidemia refers to a condition in which there are abnormally elevated levels of any or all lipids and/or lipoproteins in the blood. It is the most common form of dyslipidemia. Hyperlipidemias are generally divided into primary and secondary subtypes. Primary hyperlipidemia is generally due to genetic causes (such as a mutation in a receptor protein), while secondary hyperlipidemia arises due to other underlying causes such as diabetes or other medical conditions. Lipid and lipoprotein abnormalities are common in the general population, and are regarded as a modifiable risk factor for cardiovascular disease due to their influence on atherosclerosis.

Therefore, in accordance with further therapeutic methods and applications of the present invention described herein, the composition described herein (i.e., L-arabinose and a/the source of one or more polyphenolic compounds, such as grape marc extract) is administered in an effective amount such that hyperglycemia and/or hyperlipidemia is treated or prevented. An effective amount will be understood to be an amount which is effective to treat or prevent hyperglycemia and/or hyperlipidemia, i.e., to produce a therapeutic effect. An effective amount includes any of the doses, dosages or dosage regimens described above, each of which pertains to the amount of L-arabinose and the source of the one or more polyphenolic compounds.

The compositions of the present invention may be prepared by combining L-arabinose and a source of one or more polyphenolic compounds, e.g., grape mar extract, optionally with one or more of the other ingredients described herein, such as an additional source of dietary fibre, vitamin(s), minerals, and/or other biologically active agents, in suitable amounts to obtain a composition having the desired quantity of each component.

In certain embodiments, a mixture of the L-arabinose and source of the one or more polyphenolic compounds (e.g., grape marc extract), and optionally any one or more of the additional ingredients described herein, is prepared by mixing the L-arabinose and the source of the one or more polyphenolic compounds (e.g., grape marc extract), and optionally any one or more of the additional ingredients described herein. Such methods are well known in the art, for example, methods known in the food industry, such as those used in the preparation of health food bars and the like. This process may further comprise a forming step, wherein the mixture is moulded, pressed, spray dried or otherwise formed into a shape, e.g., a bar, ball, pellet or clusters (e.g., clusters of the type found in breakfast cereals, and the like), preferably with dimensions suitable for oral consumption by a human or other mammalian animal of the types described herein.

Besides being useful for human applications and treatments, the present invention is also useful in a range of mammals, which can also be affected by high blood sugar and/or lipid levels, obesity and weight gain. Such mammals include non-human primates (e.g. apes, monkeys and lemurs), for example in zoos, companion animals such as cats or dogs, working and sporting animals such as dogs, horses and ponies, farm animals, for example pigs, sheep, goats, deer, oxen and cattle, and laboratory animals such as rodents (e.g. rabbits, rats, mice, hamsters, gerbils or guinea pigs).

The composition as described herein may be used in a method of managing, e.g., reducing, the weight of a subject. Such methods may not alleviate or treat a treatable disorder, but rather enable a subject to maintain a healthy weight (e.g., a BMI of from 18.5-24.9), or enable an overweight subject (e.g., a subject who has a BMI of from 25.0-29.9) to reduce their weight (i.e., reduce their 25 BMI), preferably to a healthy weight, or to otherwise to reduce, minimize, ameliorate or prevent weight gain in a subject. Thus, in certain embodiments, the method of reducing weight is a cosmetic (i.e., non-therapeutic) method.

In general, a suitable daily dose of the composition will be that amount of the composition which is the lowest dose effective to produce the desired degree or type of weight management. In certain embodiments, the doses, dosages and dosage regimens described above will be suitable for the method of managing the weight of a subject. A person of ordinary skill in the art will understand that a suitable dose or dosage will typically vary from subject to subject, and will dependent on factors such as the dietary habits and severity of health conditions of the subject at the outset of administration of the composition. For example, a subject seeking to maintain a healthy weight may need to consume a lesser amount of the composition than an overweight subject seeking to reduce their weight. A subject on a high caloric diet may need to consume a higher dose of the composition. The method of managing weight may be combined with other conventional weight loss measures, such as, for example, an increase in physical activity and/or a healthy or healthier diet.

The invention will now be described in detail by way of reference only to the following non-limiting examples.

### EXAMPLES

### Example 1—the Inhibitory Composition

A composition in tablet form was prepared from the components listed in Table 1 below.

TABLE 1

| | Amount/Tablet (mg) | w/w (%) |
|---|---|---|
| Ingredients | | |
| L-Arabinose | 600.0 | 60.00 |
| Grape marc extract (GME) | 45.0 | 4.50 |
| Excipients | | |
| Diluent/filler/extender (1) | 265.2 | 26.52 |
| Diluent/filler/extender (2) | 35.0 | 3.50 |
| Lubricant | 4.8 | 0.48 |
| Cellulosic Binder | 50.0 | 5.00 |
| Total | 1000 mg | 100 |

The grape marc extract had a tannin content of about 33-35% w/w. The composition has a ratio of L-arabinose to GME of about 93:7.

Four types of enzymatic assays were conducted using a sample of the composition in tablet form as detailed in Table 1.

α-Glucosidase Inhibition

This method describes how to measure the inhibition of α-glucosidase activity. This measurement is performed in an aqueous medium. The results are expressed in mg of required product to inhibit 50% of the activity of one mg of α-glucosidase in the described experimental conditions.

The sample was crushed and dissolved at different concentrations. The enzyme reaction to inhibit is the transformation of a chromogenic substrate specific to α-glucosidase: 4-nitro-phenyl D-glucopyranosidase. Change in colour (yellow) observed and was measurable at 400 nm, when the sample was hydrolysed by the enzyme. The kinetic of this reaction was monitored by spectrophotometry and the maximum reaction rate of the enzyme was then determined, followed by calculations of percentage of inhibition. The curve representing the percentage of inhibition versus concentration sample allows the determination of the amount of product required to inhibit 50% of one mg of α-glucosidase's activity.

α-Amylase Inhibition

This method describes how to measure the inhibition of α-amylase activity. This measurement is performed in an aqueous medium. The results are expressed in mg of required product to inhibit 50% of the activity of one mg of α-amylase in the described experimental conditions.

The sample was dissolved at different concentrations. The enzyme reaction to inhibit is the transformation of a chromogenic substrate specific to α-amylase: 2-chloro,4-nitrophenyl maltotrioside. Changes in colour (yellow) observed and was measurable at 400 nm, when the sample was hydrolysed by the enzyme. The kinetic of this reaction was monitored by spectrophotometry and the maximum reaction rate of the enzyme was then determined, followed by calculations of percentage of inhibition. The curve representing the percentage of inhibition versus concentration sample allows the determination of the amount of product required to inhibit 50% of one mg of α-amylase's activity.

Lipase Inhibition Method

Lipase inhibition assay was carried out as per the method described in US2008/0317821 A1. In brief, the total reaction volume of 50 μl contained 13 mM Tris 150 mM NaCl, 1.36 mM CaCl₂ buffer (pH-8.0)/positive control/test sample at various concentrations, 0.396 U of lipase enzyme, 5 μl of de-mineralized water and 0.1 mM of substrate (4-Methyl umbelliferyloleate). The plate was mixed and the change in fluorescence was determined at 25° C. for 20 min at an excitation of 360 nm and emission of 460 nm using FLUOstar Optima (BMG Labtech, Germany). All reactions were carried out at 6 replicates. A control reaction was run without the test sample.

Trypsin Inhibition Method

Trypsin inhibition assay was carried out as per the method of Cannell et al., 1988 (“Methods in Biotechnology—Natural Products Isolation”, edited by Richard JP Cannell, Humana Press, Totowa, N.J.). In brief, pre-incubation volume contained 0.4M tris-HCl pH 7.5/positive control/test solution of various concentrations and 21.6 units enzyme. The reaction mixture was mixed and pre-incubated at 37° C. for 30 minutes. Following pre-incubation substrate (BAPNA) was added to a final concentration of 333.33 μM. The reaction mixture was mixed and incubated at 37° C. for 30 minutes. The absorbance was measured at 410 nm in a micro plate reader (VersaMax microplate reader, Molecular devices, USA). A control reaction was run without the test sample.

### Example 2—the Analytical Assays

A further series of compositions, based on the composition of the tablet shown in Table 1 but with varying ratios of L-arabinose and GME, were tested in the same way as in Example 1. These compositions and the test results are summarized in Table 2 above as Formula F2, F3, F4 and F5. Comparative tablets comprising only L-arabinose (Formula A) or GME (Formula G) were also tested.

Results are summarised in Table 2 below.

Formula F1 corresponds to the composition of the tablet shown in Table 1 above.

TABLE 2

| | A | F1 | F2 | F3 | F4 | F5 | G |
|---|---|---|---|---|---|---|---|
| Formula (F) | | | | | | | |
| L-Arabinose (%) | 100 | 93 | 77 | 46 | 23 | 30 | 0 |
| GME (%) | 0 | 7 | 23 | 54 | 77 | 70 | 100 |
| Results (IC50) | | | | | | | |
| α-Amylase Inhibition (ug/ml) | NA | 204.81 | 107.06 | 11.87 | 11.73 | 19.05 | 8.63 |
| α-Glucosidase Inhibition (ug/ml) | 471.63 | 733.53 | 627.91 | 554.14 | 1080.99 | 1013.6 | 928.02 |
| Lipase Inhibition (ug/ml) | NA | 66.36 | 14.9 | 6.68 | 1.04 | 2.22 | 1.8 |
| Trypsin Inhibition (ug/ml) | NA | 248.74 | 176.14 | 38.46 | 35.85 | 36.97 | 17.58 |

1. A composition comprising a pentose and one or more polyphenolic compounds.

2. A composition according to claim 1, wherein the one or more polyphenolic compounds are plant-derived.

3. A composition according to claim 2, wherein the one or more plant-derived polyphenolic compounds are plant-derived tannins.

4. A composition according to claim 3, wherein the plant-derived tannins are derived from the plant Vitis spp.

5. A composition according to claim 3 or 4, wherein the plant-derived tannins are comprised within grape marc extract.

6. A composition according to any preceding claim, wherein the pentose is L-arabinose.

7. A composition according to claim 6, wherein the L-arabinose is derived from the plant Zea mays.

8. A composition according to any preceding claim, wherein the composition comprises L-arabinose and grape marc extract.

9. A composition according to claim 8 wherein the grape marc extract comprises from about 25% w/w to about 40% w/w tannins.

10. A composition according to claim 8 or 9, wherein the weight ratio of L-arabinose to grape marc extract is from about 95:5 to about 40:60.

11. A composition according to claim 10, wherein the weight ratio of a L-arabinose to grape marc extract is from about 60:40 to about 40:60.

12. A composition according to any preceding claim, wherein the composition comprises at least about 20% w/w L-arabinose and/or at least about 2% w/w grape marc extract, based on the total weight of the composition.

13. A composition according to any preceding claim, further comprising dietary fibre of plant and/or non-plant origin.

14. A composition according to claim 13, wherein the dietary fibre is derived from Abelmoschus app.

15. A composition according to any preceding claim, wherein the composition is a foodstuff, a food supplement, a beverage, a beverage supplement, a dietary supplement, a health supplement, a meal replacement product, a food additive, an animal feed or a feed additive.

16. A pharmaceutical composition comprising a composition according to any one of claims 1-15 and a pharmaceutically acceptable carrier and/or excipient and/or diluent.

17. A pharmaceutical composition according to claim 16, wherein the pharmaceutically acceptable carrier and/or excipient and/or diluent is selected from one or more of binder, diluent, lubricant and coating agent.

18. A pharmaceutical composition according to claim 17, wherein the composition comprises no more than about 50% w/w pharmaceutically acceptable carrier and/or excipient and/or diluent.

19. A pharmaceutical composition according to any one of claims 16-18 in the form of an oral preparation.

20. A pharmaceutical composition according to claim 19, wherein the oral preparation is a capsule, tablet, powder, syrup, solution, suspension, sachet or shake.

21. A composition according to any one of claims 1-15 or a pharmaceutical composition according to any one of claims 16-20 for use in inhibiting an enzyme selected from one or more of α-glucosidase, α-amylase, lipase and protease.

22. An in vitro method for inhibiting an enzyme selected from one or more of α-glucosidase, α-amylase, lipase and protease, said method comprising contacting the enzyme with a composition according to any one of claims 1-15 or a pharmaceutical composition according to any one of claims 16-20.

23. An in vivo method for inhibiting an enzyme selected from one or more of α-glucosidase, α-amylase, lipase and protease, said method comprising contacting the enzyme with a composition according to any one of claims 1-15 or a pharmaceutical composition according to any one of claims 13-20.

24. A method of managing, for example, reducing blood glucose and/or lipid level in a subject, comprising administering to the subject an effective amount of a composition according to any one of claims 1-15 or a pharmaceutical composition according to any one of claims 16-20.

25. A method of managing, for example, reducing glucose and/or lipid absorption in a subject, said method comprising administering to the subject an effective amount of a composition according to any one of claims 1-15 or a pharmaceutical composition according to any one of claims 16-20.

26. A method according to claim 24 or 25, wherein the management or reduction of glucose absorption and/or blood glucose level is obtained by inhibiting the activity of α-glucosidase and α-amylase.

27. A method according to claim 24 or 25, wherein the management or reduction of lipid absorption and/or level is obtained by inhibiting the activity of lipase.

28. A method of managing, for example, reducing digestion of dietary protein, carbohydrate and/or fat and absorption of calories from said dietary protein, carbohydrate and/or fat in a subject, comprising administering to the subject an effective amount of a composition according to any one of claims 1-15 or a pharmaceutical composition according to any one of claims 16-20, wherein the management or reduction of digestion and absorption of dietary protein is obtained by inhibiting the activity of protease, and/or wherein management or reduction of digestion and absorption of dietary carbohydrate is obtained by inhibiting the activity of α-glucosidase and α-amylase, and/or wherein management or reduction of digestion and absorption of dietary fat is obtained by inhibiting the activity of lipase.

29. A method according to any one of claims 23-28, wherein the composition is administered to the subject from about 15 minutes to about 60 minutes before or after a meal.

30. A composition according to any one of claims 1-15 or a pharmaceutical composition according to any one of claims 16-20 for use in managing, for example, reducing glucose and/or lipid absorption in a subject.

31. A composition according to any one of claims 1-15 or a pharmaceutical composition according to any one of claims 16-20 for use in managing, for example, reducing blood glucose and/or lipid level in a subject.

32. A composition according to any one of claims 1-15 or a pharmaceutical composition according to any one of claims 16-20 for use in managing, for example, reducing digestion of dietary protein, carbohydrate and/or fat and absorption of calories from said dietary protein, carbohydrate and/or fat in a subject.

33. A non-therapeutic method of reducing the body weight of a subject, said method comprising administering to the subject an effective amount of a composition according to any one of claims 1-15 or a pharmaceutical composition according to any one of claims 16-20.

34. A composition according to any one of claims 1-15 or a pharmaceutical composition according to any one of claims 16-20 for use in therapeutically reducing the body weight of a subject.

35. A method of reducing the caloric absorption from dietary fats, carbohydrates and/or proteins in a subject, said method comprising administering to the subject an effective amount of a composition according to any one of claims 1-15 or a pharmaceutical composition according to any one of claims 16-20.

36. A composition according to any one of claims 1-15 or a pharmaceutical composition according to any one of claims 16-20 for use in reducing the caloric absorption from dietary fats, carbohydrate and/or proteins in a subject.

37. A method of treating or preventing obesity and/or metabolic syndrome in a subject, said method comprising administering to the subject an effective amount of a composition according to any one of claims 1-15 or a pharmaceutical composition according to any one of claims 16-20.

38. A method of treating or preventing hyperglycemia and/or hyperlipidemia in a subject, said method comprising administering to the subject an effective amount of a composition according to any one of claims 1-15 or a pharmaceutical composition according to any one of claims 16-20.

39. A composition according to any one of claims 1-15 or a pharmaceutical composition according to any one of claims 16-20 for use in treating or preventing obesity and/or metabolic syndrome in a subject.

40. A composition according to any one of claims 1-15 or a pharmaceutical composition according to any one of claims 16-20 for use in treating or preventing hyperglycemia and/or hyperlipidemia in a subject.

41. Use of a composition according to any one of claims 1-15 or a pharmaceutical composition according to any one of claims 16-20 in the manufacture of a medicament for managing, for example, reducing glucose and/or lipid absorption in a subject, or for managing, for example, reducing blood glucose and/or lipid level in a subject, or for treating or preventing obesity and/or metabolic syndrome in a subject, or for treating or preventing hyperglycemia and/or hyperlipidemia in a subject.

42. Use of a composition according to any one of claims 1-15 or a pharmaceutical composition according to any one of claims 16-20 in the manufacture of a medicament for managing, for example, reducing digestion of dietary protein, carbohydrate and/or fat and absorption of calories from said dietary protein, carbohydrate and/or fat in a subject.
